(19) United States
(12) Patent Application Publication
(10) Pub. No.: US 2007/0269385 A1
(43) Pub. Date: Nov. 22, 2007

(54) DEVICES, METHODS, AND SYSTEMS FOR DELIVERING THERAPEUTIC AGENTS FOR THE TREATMENT OF SINUSITIS, RHINITIS, AND OTHER DISORDERS

(75) Inventors: Anthony Joon Yun, Palo Alto, CA (US); Kirk Patrick Seward, Dublin, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Mercator Medsystems, Inc, San Leandro, CA (US)

(21) Appl. No.: 11/748,975

(22) Filed: May 15, 2007

Related U.S. Application Data
(60) Provisional application No. 60/820,725, filed on Jul. 28, 2006.
(60) Provisional application No. 60/747,557, filed on May 18, 2006.

(51) Int. Cl. A61K 31/7048; A61K 31/7034; A61K 39/395; A61K 31/496; A61K 31/366; A61K 31/22; A61K 31/65; A61K 31/33; A61K 9/12; A61K 31/34

(52) U.S. Cl. 424/45; 514/152; 514/35; 514/36; 514/28; 514/183; 514/312; 514/253.08; 514/471; 514/423; 514/460; 514/548; 424/133.1

(57) ABSTRACT

Methods and kits for delivering pharmaceutical agents to the sinuses, sinus ostia, Eustachian tube, and pharynx are presented. A needle tip is translated through the mucosal tissue layer to a sub-epithelial or peri-luminal orientation and pharmaceutical agents are delivered into the sub-epithelial or peri-luminal tissue. Drugs distribute from the site of infusion to treat conditions including sinusitis and allergic rhinitis, among others.

### CROSS-REFERENCES TO RELATED APPLICATIONS

This application claims the benefit of prior provisional application Nos. 60/820,725 (Attorney Docket No. 0021621-002600US), filed on Jul. 28, 2006, and 60/747,557 (Attorney Docket No.: 021621-002400US), filed on May 18, 2007, the full disclosures of which are incorporated herein by reference.

### BACKGROUND OF THE INVENTION

### Field of the Invention

The present invention relates generally to medical methods and devices. More particularly, the present invention relates to methods and systems for delivering anti-inflammatory and other agents into sub-epithelial or peri-luminal tissue surrounding a patient's sinus structures for treatment of sinus disease.

The paranasal sinuses are air-filled cavities within the facial skeleton. The paranasal sinuses include the frontal sinuses, ethmoid sinuses, maxillary sinuses, and sphenoidal sinuses. The paranasal sinuses are lined with mucous-producing epithelial tissue. Each paranasal sinus is contiguous with a nasal cavity and drains mucous into the nasopharynx through a sinus ostium. Although other factors may be involved, the development of sinusitis (inflammation of the mucosal lining of the sinuses) is most often attributed to blockage of one or more of these sinus ostia, followed by mucostasis, potential damage to the epithelial lining, reduced oxygen tension, and microbial overgrowth in the sinus cavity. Ostial blockage may stem from predisposing anatomical factors, or inflammation and edema of the mucous lining in the area of the ostia, arising from such etiologies as viral or bacterial infection, fungus, chronic allergic processes, or combinations thereof.

Traditionally, sinusitis has been medically managed by the oral administration of anti-infective agents and steroids. However, chronic use of such agents risks favoring selection of agent-resistant populations of organisms which can then lead to perpetuation of inflammation. The use of localized delivery of anti-inflammatory agents, anti-stress agents, and anti-infective agents, at concentrations which provide anti-inflammatory benefits without promoting the growth of agent-resistant organisms, may provide significant medical benefits for patients afflicted with sinusitis. Additionally, agents may be used which if delivered systemically and/or over extended time periods, might cause side effects. For the purpose of definition, agents meeting these criteria will be referenced as therapeutic agents.

Localized delivery of therapeutic agents into the sinuses has taken the form of inhaled mists, topical drops, creams and gels, or solid implants that elute drug slowly over time. The drawback of each of these systems arises from their inability to penetrate the sinus mucosa and relieve edematous conditions arising from sub-epithelial (just below the skin lining the sinus cavities and ostia) or peri-luminal etiology/pathology. Inhaled mist-based systems or drops typically only penetrate the nasal cavity and do not move deep into the blocked sinuses. Local, topical delivery of either agents alone or solid implants that elute drugs is cumbersome and time consuming, and is not justified due to their lack of success in penetrating to the underlying disease process causing the sinusitis.

For example, U.S. Patent Application Publication 2004/0116958A1 (Gopferich et al.) describes a tubular sheath or “spacer” formed of biodegradable or non-biodegradable polymer that, prior to insertion in the patient's body, is loaded with a controlled amount of an active substance, such as a corticosteroid or anti-proliferative agent. Surgery is performed to create a fenestration in a frontal sinus and the sheath is inserted into such fenestration. Thereafter, the sheath which has been preloaded with the active substance is inserted into the surgically created fenestration where it a) deters closure of the surgically created fenestration, b) serves as a conduit to facilitate drainage from the sinus and c) delivers the active substance. The sheath of said application remains in the sinus and in contact with the sinus mucosa without penetrating beyond the epithelium. Thus, while drugs may be delivered or eluted from the sheath, direct sub-epithelial or peri-luminal delivery is not accomplished.

Other publications have also reported that introduction of drugs directly into the paranasal sinuses is effective in the treatment of sinusitis. For example, refer to Tarasov D I, et al., “Application of Drugs Based on Polymers in the Treatment of Acute and Chronic Maxillary Sinusitis”, Vestn Otorinolaringol. 1978;6:45-47. Also, Deutschmann R, et al., “A Contribution to the Topical Treatment of [Maxillary] Sinusitis Preliminary Communication,” Stomat. 1976; DDR26:585-92 describes the placement of resorbable drug delivery depot within the maxillary sinus for the purpose of eluting drugs, specifically Chloramphenicol. In this clinical series a water soluble gelatin was used as carrier and was mixed with the drug prior to application and introduced as a mass into the sinus. Since the substance had little mechanical integrity and dissolved in a relatively short timeframe, to achieve a therapeutic effect, the author suggested that it must be instilled every 2 to 3 days. An alternative to gelatin could be a sponge loaded with the therapeutic substance as described by Jacobsen et al. in U.S. Pat. No. 6,398,758. In this patent directed at delivering a sustained release device against the wall of a blood vessel, a hollow cylindrical sponge is loaded with drug and pressed against the wall. This allows the drug to contact the wall while sustaining flow within the central lumen. Further, a skin is provided to direct the drug into the walls of the vessel and prevent drug from flowing back into the lumen. While sponges loaded with drug at the time of their application do permit some degree of sustained release, the time required to load them correlates closely with the time over which they will elute the substance. Thus, if delivery is required for a longer period of time (such as to penetrate the sinus mucosa and epithelium, additional mechanisms must be employed either to regulate the release of agents or facilitate the more directed administration of agents beyond the mucosa and epithelium.

There are also several examples in the patent literature where various sustained release mechanisms or intra-sinus delivery methods have been proposed using systems with pre-incorporated drugs into matrices or polymers, or systems to temporarily occlude sinus ostia for the flushing of drug into the sinus cavities. These include U.S. Pat. No. 3,948,254 (Zafferoni), U.S. 2003/0185872A2 (Kochinke), WO 92/15286 (Shikani), U.S. Pat. No. 5,512,055 (Domb, et al.), and U.S. 2005/0245906A1 (Makower, et al.) and U.S. 2006/0106361A1 (Muni et al.). In general, these references discuss the various materials and structures that may be used for intrasinus delivery of therapeutic agents. These references, however, do not describe any form of sub-epithelial or periluminal delivery of therapeutic or diagnostic agents in the paranasal sinuses or sinus ostia or other locations in the body useful for the treatment of sinusitis or other conditions. Balloon catheters can be introduced to and inflated within the sinuses for “sinuplasty” and other purposes, as taught by U.S. Pat. No. 6,607,546 B1 (Murken) and U.S. 2006/0149310A1 (Becker).

There remains a need in the art for the development of new devices and methods to deliver drugs or other therapeutic or diagnostic agents directly beyond the epithelium of the paranasal sinuses and sinus ostia or other locations in the body for the treatment of sinusitis or other diseases and disorders.

### BRIEF SUMMARY OF THE INVENTION

The present invention provides devices and methods for the delivery of agents including anti-inflammatory agents, anti-stress agents, and/or an anti-infective agents at a sub-antimicrobial concentration, to sub-epithelial or peri-luminal tissue surrounding a paranasal sinus or other body lumen. Delivery is accomplished via trans-mucosal, sub-epithelial or peri-luminal penetration (injection or infusion) using an infusion and/or injection catheter. Infusion catheters may have one or more ports or pores through which streams of agents may be directed under high pressure to penetrate the mucosa and epithelium. Catheters may alternatively or additionally include one or more microneedle penetration members that, when placed into the sinus or ostium and deployed, may position an injection port trans-mucosally and into a sub-epithelial or peri-luminal orientation prior to infusion or injection.

Anti-inflammatory agents, anti-stress agents, and anti-infective agents are delivered into a paranasal sinus or other sub-epithelial or peri-luminal sinus or nasopharynx tissue for the prophylaxis or treatment of sinusitis, rhinitis, or other diseases of the nose, sinus, or pharynx. The agents are typically delivered by catheter, usually being introduced trans-mucosally and sub-epithelially into the peri-luminal tissue surrounding paranasal sinuses or sinus ostia. Anti-infective agents may be delivered at a “sub-antimicrobial” concentration, that is, a concentration that does not inhibit microbial growth. Specific to the invention is the use of a microneedle injection/infusion catheter for delivery of said agents.

The anti-inflammatory agent and/or anti-stress agent, and/or the subantimicrobial concentration of the anti-infective agent may be used in a system for treating sinusitis, rhinitis, or other diseases of a body lumen selected from the sinus, nasal, or Eustachian lumens and cavities. The anti-inflammatory agent, anti-stress agent, and/or sub-antimicrobial concentration of the anti-infective agent may also be used for reducing inflammation resulting from a sinus procedure.

Treatments according to the present invention may comprise a single injection or infusion, or may comprise multiple injections or infusions over a period of hours, days, weeks, or longer. A single injection or infusion may comprise one or more boluses of the agent being delivered, with individual boluses being in the range from 0.01 ml to 5 ml, typically being from 0.1 ml to 1 ml.

A particular advantage of the present invention is the ability to deliver a wide variety of agents widely throughout the sinus and peri-luminal sinus tissue with only one or a limited number of injections. It is presently believed that such wide distribution of the drug is best achieved when the drug is delivered into the peri-luminal sinus tissue beyond or within the sinus mucosa and beneath the epithelial membrane. The thickness of the sinus mucosa can vary depending on anatomy and state of disease, but is typically in the range of 0.1 mm to 5 mm.

It is further believed that wide distribution and retention of agents in the sinus mucosa may result from entry of the agent into the sub-epthelial space of the sinus tissue. While this understanding of the potential mechanism of action may help understand and define the present invention, the present invention in no way depends on the accuracy of understanding this mechanism of distribution.

The methods and systems of the present invention preferably utilize injection from an intra-luminal device such as an intra-sinus or intra-Eustachian catheter in order to deliver the therapeutic agents to the peri-sinus space as defined above. Use of intra-luminal delivery approach is particularly preferred as access is provided to deep recesses of the sinus without otherwise more invasive procedures involving sinusotomy. One such direct access is provided, however, the methods of the present invention may be performed by injection by a needle through a sinusotomy, for example. Accurate positioning of the needle may be achieved using, for example, fluoroscopic imaging, endoscopic imaging, or the like.

In particular, the preferred intra-luminal injection devices and methods of the present invention comprise a device and method for injecting a therapeutic concentration of an agent into the peri-sinus tissue by advancing a needle from a lumen of the sinus or Eustachian tube to the target location beyond the sinus mucosa and epithelium. The therapeutic concentration of agent is then delivered through the needle to the target tissue. The needle is at least into the peri-sinus tissue beneath the epithelium of the sinus cavity or lumen.

In another aspect of this invention, agents can be directly injected or infused into the nasal turbinates for the treatment of inflammation. The nasal turbinates may be accessed and injected from an intranasal approach with a similar intra-luminal catheter as that described above.

In yet another aspect of this invention, agents can be directly injected or infused into sinus polyps for reduction of polyp number, density, or volume in a patient with polyposis. Sinus polyps may also be accessed and injected from an intranasal approach with a similar intra-luminal catheter as that described above.

The therapeutic agents will be injected or infused under conditions and in an amount sufficient to permeate circumferentially around the peri-sinus space of the sinus cavity, ostium or tube or into sinus polyps over an axial length of at least about 5 mm, usually at least about 1 cm, and more usually greater than 1 cm. Thus, the needle may be advanced in a radial direction to a depth in the tissue or polyps surrounding the cavity, lumen, or tube typically by a depth greater than 0.2 mm and more typically in a range of 0.5 mm to 3 mm.

Systems according to the present invention for treating a patient suffering from sinusitis or rhinitis or other diseases or inflammatory conditions of the sinus or peri-Eustachian tissue comprise an amount of therapeutic drug, particularly an anti-inflammatory drug or antibiotic agent or anti-stress agent or anti-infective agent, sufficient to treat the inflamed or diseased tissue, and an intra-luminal catheter having a needle adapted for injecting the drug into a location beyond the epithelium of the sinus cavity or ostium or Eustachian tube as described above.

### BRIEF DESCRIPTION OF THE DRAWINGS

FIG. 1A is a schematic, perspective view of an intra-luminal injection catheter suitable for use in the methods and systems of the present invention.

FIG. 1B is a cross-sectional view along line 1B-1B of FIG. 1A.

FIG. 1C is a cross-sectional view along line 1C-1C of FIG. 1A.

FIG. 2A is a schematic, perspective view of the catheter of FIGS. 1A-1C shown with the injection needle deployed.

FIG. 2B is a cross-sectional view along line 2B-2B of FIG. 2A.

FIG. 3 is a schematic, perspective view of the intravascular catheter of FIGS. 1A-1C injecting drug into peri-sinus tissue surrounding a sinus cavity, ostium, or tube in accordance with the methods of the present invention.

FIG. 4 is a schematic, perspective view of another embodiment of an intra-luminal injection catheter useful in the methods of the present invention.

FIG. 5 is a schematic, perspective view of still another embodiment of an intra-luminal injection catheter useful in the methods of the present invention, as inserted into a patient's sinuses.

FIGS. 6A and 6B are schematic views of other embodiments of an intra-luminal injection catheter useful in the methods of the present invention (in an unactuated condition) including multiple needles.

FIG. 7 is a schematic view of yet another embodiment of an intra-luminal injection catheter useful in the methods of the present invention (in an unactuated condition).

FIG. 8 is a perspective view of a needle injection catheter useful in the methods and systems of the present invention.

FIG. 9 is a cross-sectional view of the catheter FIG. 8 shown with the injection needle in a retracted configuration.

FIG. 10 is a cross-sectional view similar to FIG. 9, shown with the injection needle laterally advanced into sub-epithelial or peri-luminal tissue for the delivery of drug according to the present invention.

FIG. 11 is a cross-sectional view of human frontal and maxillary sinus cavities, shown with a guidewire placed intra-luminally to provide access to a frontal sinus FS and sinus ostium SO for sub-epithelial or peri-luminal treatments.

FIG. 12 is a cross-sectional view similar to FIG. 11 shown with the sub-epithelial or peri-luminal treatment catheter C in place in the frontal sinus ostium, and injecting a drug D to the sub-epithelial or peri-luminal tissue.

FIG. 13 is a cross-sectional view of the human Eustachian tube EuT, middle ear ME, and external auditory canal EAC with a guidewire placed intra-luminally to provide access to the Eustachian tube for sub-epithelial or peri-luminal treatments.

FIG. 14 is a cross-sectional view similar to FIG. 13 shown with the sub-epithelial or peri-luminal treatment catheter C in place in the Eustachian tube and injecting a drug D to the sub-epithelial or peri-luminal tissue.

### DETAILED DESCRIPTION OF THE INVENTION

The present invention provides devices, methods, and systems for treating patients at risk of or suffering from sinusitis, rhinitis, or other diseases of the sinus, nasal, or Eustachian lumens or cavities. In particular, these patients will have been diagnosed or otherwise determined to be suffering from an inflammation or infection (typically bacterial, viral, or fungal in origin) of the naso-sinus or Eustachian tube. In other cases, patients who have recently had a sinus procedure typically employed to open a blocked sinus suffer from the inflammatory reaction of the body to the procedure, and may be candidates for receiving treatment according to the present invention in order to reduce inflammation, swelling, and risk of infection.

The acceptable promicrobial concentration of any anti-inflammatory and/or anti-stress agent, and/or the subantimicrobial concentration of any anti-infective agent, would be determined via standard laboratory assays, such as minimal inhibitory concentration (MIC). Prior art as to the determination of said concentrations are also described in U.S. RE 34656.

The methods of delivery of an agent in accordance with the principles of the present invention may take various forms, but are generally designed to have characteristics appropriate for the intended method of delivery, e.g., through the sinus ostium or by puncture through a sinus wall. Injection or infusion using a microneedle catheter is described generally in U.S. patent application Ser. Nos. 09/961,079; 09/961,080; 10/490,129 and 10/490,191 and U.S. Pat. Nos. 6,547,803 and 6,860,867, which describe microneedle catheters and methods of use. U.S. Pat. No. 4,578,061 describes needle injection catheters having deflectable, axially advanceable needles. U.S. Pat. No. 5,538,504 describes a needle injection catheter having a transversely oriented needle that is laterally advanced by a balloon driver. Also of interest are U.S. Pat. Nos. 6,319,230; 6,283,951; 6,283,947; 6,004,295; 5,419,777; and 5,354,279. U.S. patent application Nos. 10/350,314; 10/610,790; 10/728,186; 10/691,119; 10/393,700; 10/824,768 are of common invention and assignment as this application and describe devices and methods for perivascular (peri-luminal) agent delivery, the entire disclosure of which are incorporated herein by reference.

For purposes of this description, we use the following terms as defined in this section, unless the context of the word indicates a different meaning.

The term “sinus” is meant to refer to all sinuses, i.e., the maxillary, ethmoid, frontal, and sphenoidal sinuses, as well as to the lumens leading to each of the sinus cavities and nasopharynx.

The term “lumen” is meant to refer to an opening, whether a cavity, tube, or other potential space, typically distinguished from the “peri-lumen” by a change in structure.

The term “peri-luminal” is meant to refer to the potential space near the lumen, but outside the border defined by the boundary between “lumen” and “lumen wall”. The term “peri-luminal” is meant to include the epithelium and sub-epithelial tissue, in the case that an epithelium exists.

The term “epithelium” is meant to refer to the membranous tissue composed of one or more layers of cells separated by very little intercellular substance and forming the covering of most internal and external surfaces of the body and its organs. In the case of the paranasal sinuses, the epithelium may act as a border between tissue and lumens of the sinuses. The term “sub-epithelial” refers to the potential space within the tissue and beneath (or beyond) the epithelium.

The term “subject” is meant to refer to all mammalian subjects, preferably humans.

Mammals include, but are not limited to, primates, farm animals, sport animals, cats, dogs, rabbits, mice, and rats.

The terms “treat”, “treating”, or “treatment” are meant to refer to the resolution, reduction, or prevention of sinusitis, rhinitis or the sequelae of sinusitis or rhinitis.

As used herein, the terms “agent” and “drug” are used interchangeably and refer to any substance used to treat sinusitis, rhinitis, or other diseases of the sinus or Eustachian tissue.

The term “sub-antimicrobial concentration” is meant to refer to a concentration of anti-infective agent that does not produce toxic effects on or reduction in the growth of the target organism against which it is customarily directed.

The term “anti-infective agents” generally includes antibacterial agents, antifungal agents, antiviral agents, and antiseptics.

Examples of antibacterial agents that may be used at sub-antimicrobial concentrations include aminoglycosides, amphenicols, ansamycins, lactams, lincosamides, macrolides, nitrofurans, quinolones, sulfonamides, sulfones, tetracyclines, and any of their derivatives. In one variation, tetracyclines are the preferred antibacterial agents. The tetracyclines that may be used include tetracycline itself, doxycycline, and minocycline.

Examples of antifungal agents that may be used at subantimicrobial concentrations include allylamines, imidazoles, polyenes, thiocarbamates, triazoles, and any of their derivatives. In one variation, imidazoles are the preferred antifungal agents.

Examples of anti-inflammatory and anti-stress agents that may be used include, but are not limited to: interferon alpha-2a, interferon alpha-2b, interferon beta-1a, interferon beta-1b, interferon gamma, and the like; rituximab, adalimumab, infliximab, alefacept, etanercept, and the like; atorvastin, fluvastatin, lovastatin, mevastatin, pravastatin, rosuvastatin, simvastatin, and the like; fenofibrate; gemfibrozil; niacin; niacinamide; nicotine; diphenhydramine, triprolidine, tripelenamine, fexofenadine, chlorpheniramine, doxylamine, cyproheptadine, meclizine, promethazine, phenyltoloxamine, hydroxyzine, brompheneramine, dimenhydrinate, cetirizine, loratadine, and the like; acrivastine, brompheniramine, clemastine; acarbose, glimepride, glyburide, metform, miglitol, pioglitazone, repaglinide, rosiglitazone, and the like; aspirin, salicylic acid, salsalate, diflunisal, ibuprofen, indomethacin, oxaprozin, sulindac, ketorolac, ketoprofen, nabumetone, piroxicam, naproxen, diclofenac, celecoxib, rofecoxib, valdecoxib, and the like; cyclosporine, tacrolimus, pimecrolimus, and the like; levamisole; mycophenolate mofetil; methotrexate; cyclophosphamide; azathioprine; hydroxychloroquine; aurothioglucose; auranofin; penicillamine; sulfasalazine; leflunomide; sirolimus; paclitaxel, docetaxel, and the like; botulinum toxin; atenolol, betaxolol, bisoprolol, carvedilol, esmolol, labetalol, metoprolol, nadolol, pindolol, propanolol, sotalol, timolol, and the like; bethanechol, oxotremorine, methacholine, cevimeline, carbachol, galantamine, arecoline, and the like; muscarine; pilocarpine; edrophonium, neostigmine, donepezil, tacrine, echothiophate, diisopropylfluorophosphate, demecarium, pralidoxime, galanthamine, tetraethyl pyrophosphate, parathion, malathion, isofluorophate, metrifonate, physostigmine, rivastigmine, abenonium acetylchol, carbaryl acetylchol, propoxur acetylchol, aldicarb acetylchol, and the like; amlodipine, diltiazem, felodiipine, isradipine, nicardipine, nifedipine, nisoldipine, verapamil, and the like; moricizine, propafenone, encainide, flecainine, tocainide, mexilietine, phenytoin, lidocaine, disopyramine, quinidine, procainamide, and the like; mifepristone; guanadrel, guanethidine, reserpine, mecamylamine, hexemethonium, and the like; hydralazine; minoxidil; labetalol, carvedilol, and the like; doxazosin, prazosin, terazosin, and the like; L-arginine; nitroglycerine, isosorbide, mononitrate, dinitrate, tetranitrate, and the like; vardenafil, tadalafil, sildenafil, and the like; spironolactone, eplerenone, and the like; candesartan, irbesartan, losartan, telmisartin, valsartan, eprosartan, and the like; benazepril, captopril, enalapril, fosinopril, lisinopril, moexipril, quinapril, ramipril, trandolapril, and the like; resinoferatoxin; alpha-bungarotoxin; tetrodotoxin; relaxin; aliskiren.

Examples of anti-inflammatory corticosteroids that may be used include, but are not limited to: triamcinolone, triamcinolone acetonide (kenalog), dexamethasone, hydrocortisone, methyl prednisolone, betamethasone, and the like.

The variations of this invention may further include components such as preservatives, buffers, binders, disintegrants, lubricants, and any other excipients necessary to maintain the structure and/or function of the anti-infective agents.

Furthermore, the agents may be placed in a pharmaceutically acceptable carrier for purposes of delivery. Common bases include, but are not limited to, carbomer, liquid paraffin, water, glycerol, propylene glycol, hyaluronic acid or sodium hyaluronate, or a combination thereof.

The agents may be used to treat sinusitis or rhinitis affecting one or more of the maxillary sinus, the frontal sinus, the ethmoidal sinus, and the sphenoidal sinus, the ostia of those sinuses or the tissue of the nasal turbinates.

Furthermore, the agents may be used to treat acute or chronic sinusitis or rhinitis arising from predisposing anatomical conditions, chronic allergic processes, or conditions related to infection by various pathogens (e.g., bacteria, fungi, and viruses).

The agents may also be used to reduce inflammation resulting from a sinus procedure, typically, a sinus drainage procedure. Examples of sinus drainage procedures include, but are not limited to, widening/enlargement of a narrowed ostium, antral puncture and washout, and intranasal antrostomy. The agents may be delivered into a sinus after the procedure is completed, but they can also be delivered into a sinus before the procedure or during the procedure.

The present invention will preferably utilize microfabricated devices and methods for sub-epithelial or peri-luminal injection of drug. The following description provides several representative embodiments of microfabricated needles (microneedles) and macroneedles suitable for the delivery of the drug into a sub-epithelial or peri-luminal space or paranasal sinus tissue. The peri-luminal space is the potential space near the lumen, but outside the border defined by the boundary between “lumen” and “lumen wall” of a paranasal sinus or Eustachian tube. The microneedle is usually inserted substantially normal to the wall of a lumen to eliminate as much trauma to the patient as possible. Until the microneedle is at the site of an injection, it is positioned out of the way so that it does not scrape against the paranasal sinus mucosa or Eustachian tube wall with its tip. Specifically, the microneedle remains enclosed in the walls of an actuator or sheath attached to a catheter so that it will not injure the patient during intervention or the physician during handling. When the injection site is reached, movement of the actuator along the lumen is terminated, and the actuator is operated to cause the microneedle to be thrust outwardly, substantially perpendicular to the central axis of a lumen, for instance, in which the catheter has been inserted.

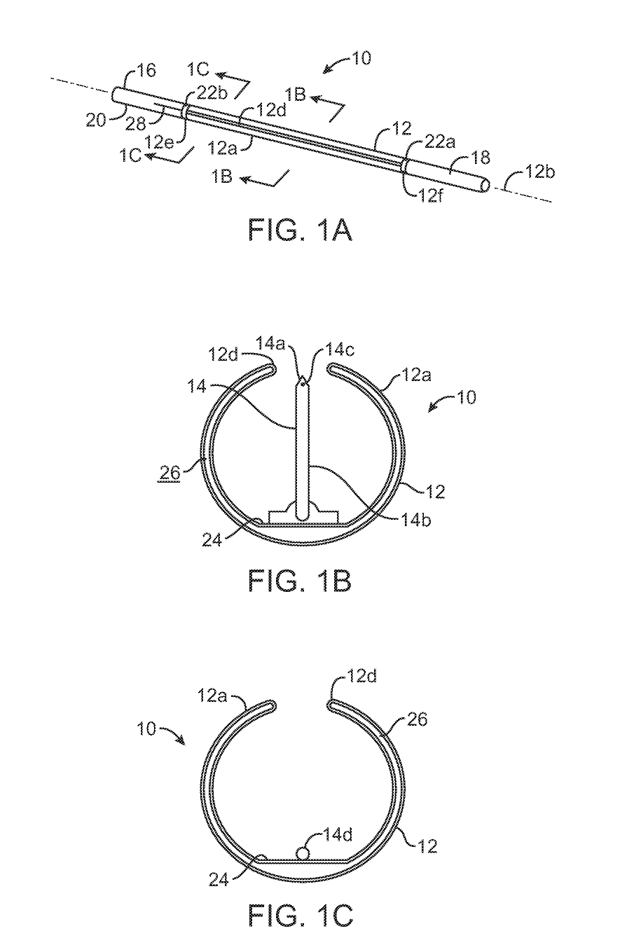

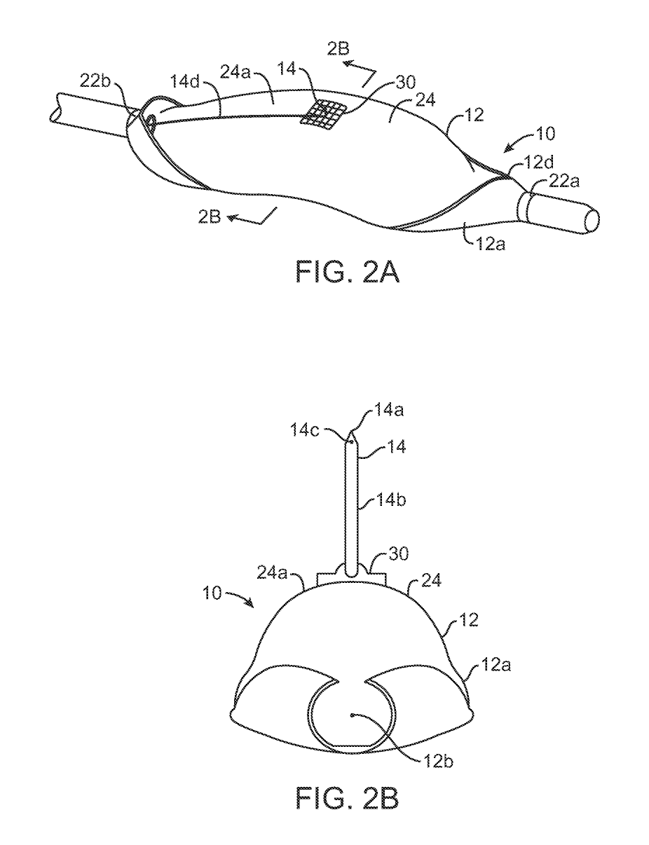

As shown in FIGS. 1A-2B, a microfabricated intra-luminal catheter 10 suitable for use in the methods of the present invention is described in U.S. Pat. No. 6,547,803, and includes an actuator 12 having an actuator body 12a and central longitudinal axis 12b. The actuator body more or less forms a U-shaped outline having an opening or slit 12d extending substantially along its length. A microneedle 14 is located within the actuator body, as discussed in more detail below, when the actuator is in its unactuated condition (furled state) (FIG. 1B). The microneedle is moved outside the actuator body when the actuator is operated to be in its actuated condition (unfurled state) (FIG. 2B).

The actuator may be capped at its proximal end 12e and distal end 12f by a lead end 16 and a tip end 18, respectively, of a therapeutic catheter 20. The catheter tip end serves as a means of locating the actuator inside a target sinus or other body lumen by use of a radio opaque coatings or markers. The catheter tip also forms a seal at the distal end 12f of the actuator. The lead end of the catheter provides the necessary interconnects (fluidic, mechanical, electrical or optical) at the proximal end 12e of the actuator.

Retaining rings 22a and 22b may be located at the distal and proximal ends, respectively, of the actuator, though their presence is not necessary for appropriate actuation given ideal or near-ideal rigidity of the actuator material. The catheter tip is joined to the retaining ring 22a, while the catheter lead is joined to retaining ring 22b. The retaining rings are made of a thin, on the order of 10 to 100 microns (μm), substantially rigid material, such as Parylene (types C, D or N), or a metal, for example, aluminum, stainless steel, gold, titanium or tungsten. The retaining rings or simple rigidity of the structure by itself forms a rigid substantially “C” or “U”-shaped structure at each end and in the center of the actuator. The catheter may be joined to the retaining rings by, for example, a butt-weld, an ultra sonic weld, integral polymer encapsulation or an adhesive such as an epoxy or cyanoacrylate.

The actuator body further comprises a central, expandable section 24 located between the rigid ends or retaining rings 22a and 22b. The expandable section 24 includes an interior open area 26 for rapid expansion when an activating fluid is supplied to that area. The central section 24 is made of a thin, semi-rigid or rigid, expandable material, such as a polymer, for instance, Parylene (types C, D or N), silicone, polyurethane or polyimide. The central section 24, upon actuation, is expandable somewhat like a balloon-device.

The central section is capable of withstanding pressures of up to about 100 psi upon application of the activating fluid to the open area 26. The material from which the central section is made of is rigid or semi-rigid in that the central section returns substantially to its original configuration and orientation (the unactuated condition) when the activating fluid is removed from the open area 26. Thus, in this sense, the central section is very much unlike a balloon which has no inherently stable structure.

The open area 26 of the actuator is connected to a delivery conduit, tube or fluid pathway 28 that extends from the catheter's lead end to the actuator's proximal end. The activating fluid is supplied to the open area via the delivery tube. The delivery tube may be constructed of Teflon® or other inert plastics. The activating fluid may be a saline solution, a radio-opaque dye, or some combination of the two.

The microneedle 14 may be located approximately in the middle of the central section 24. However, as discussed below, this is not necessary, especially when multiple microneedles are used. The microneedle is affixed to an exterior surface 24a of the central section. The microneedle is affixed to the surface 24a by an adhesive, such as cyanoacrylate.

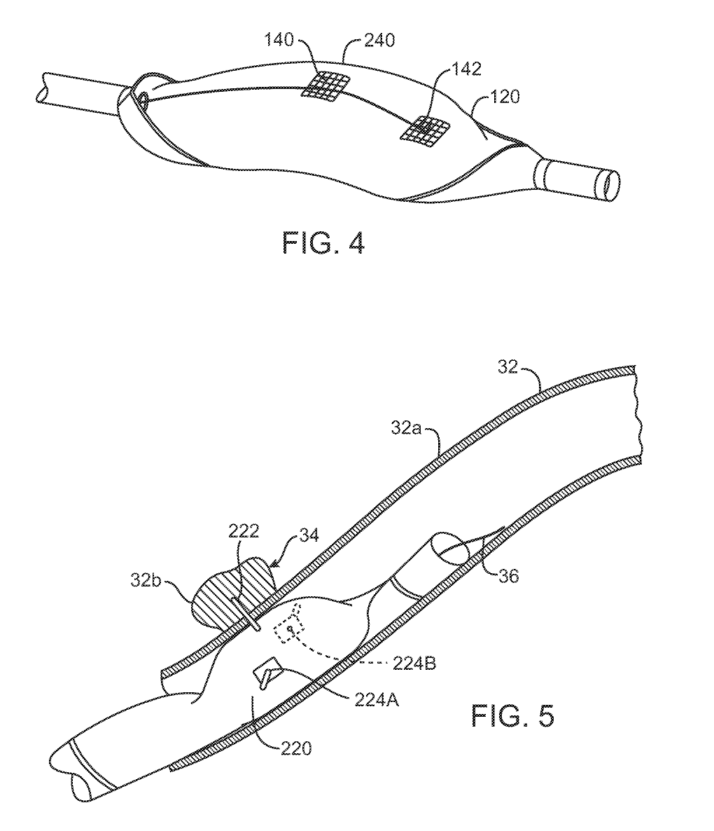

Alternatively, the microneedle maybe joined to the surface 24a by a metallic or polymer mesh-like structure 30 (See FIG. 4F), which is itself affixed to the surface 24a by an adhesive. The mesh-like structure may be-made of, for instance, steel or nylon. The microneedle may alternatively be affixed to a tube which is otherwise adhered to the surface 24a by adhesive, encapsulation bonding, or is simply a feature of the surface 24a.

The microneedle includes a sharp tip 14a and a shaft 14b. The microneedle tip can provide an insertion edge or point. The shaft 14b can be hollow and the tip can have an outlet port 14c, permitting the injection of the agent into the sub-epithelial or peri-luminal tissues.

As shown, the microneedle extends approximately perpendicularly from surface 24a. Thus, as described, the microneedle will move substantially perpendicularly to an axis of a lumen into which has been inserted, to allow direct puncture or breach of tissue walls surrounding the lumen, such as the epithelium and paranasal sinus mucosa.

The microneedle further includes a pharmaceutical or drug supply conduit, tube or fluid pathway 14d which places the microneedle in fluid communication with the appropriate fluid interconnect at the catheter lead end. This supply tube may be formed integrally with the shaft 14b, or it may be formed as a separate piece that is later joined to the shaft by, for example, an adhesive such as an epoxy.

The needle 14 may be a 30-gauge, or smaller, steel needle. Alternatively, the microneedle may be microfabricated from polymers, other metals, metal alloys or semiconductor materials. The needle, for example, may be made of Parylene, silicon or glass.

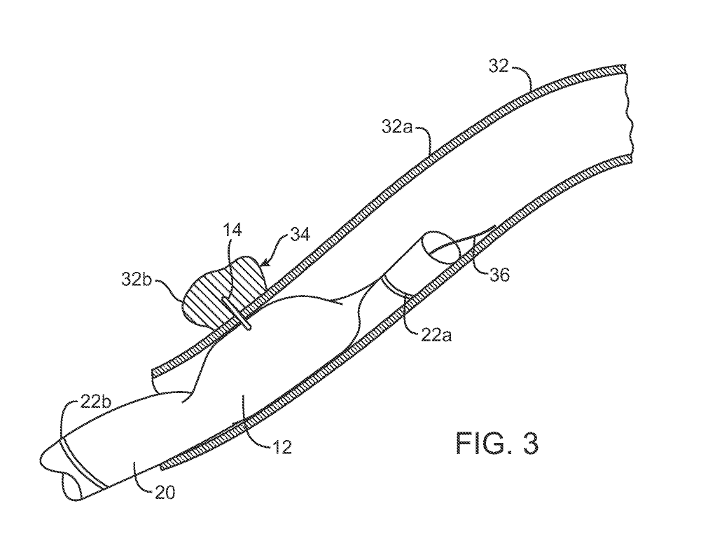

The catheter 20, in use, is inserted into a patient's body lumens, for instance, through a nostril into a paranasal sinus ostium 32, until a specific, targeted region 34 is reached (see FIG. 3). The targeted region 34 may be at or proximate to the site of tissue damage or inflammation, typically being within 100 mm or less to allow migration of the therapeutic agents. As is well known in catheter-based interventional procedures, the catheter 20 may follow a guide wire 36 that has previously been inserted into the patient. Optionally, the catheter 20 may also follow the path of a previously-inserted guide catheter (not shown) that encompasses the guide wire. The catheter may instead be inserted under the aid of endoscopic guidance, using a floppy-tipped catheter to minimize trauma.

During maneuvering of the catheter 20, well-known methods of fluoroscopy, endoscopy, or magnetic resonance imaging (MRI) can be used to image the catheter and assist in positioning the actuator 12 and the microneedle 14 at the target region. As the catheter is guided inside the patient's body, the microneedle remains unfurled or held inside the actuator body so that no trauma is caused to the body lumen walls.

After being positioned at the target region 34, movement of the catheter is terminated and the activating fluid is supplied to the open area 26 of the actuator, causing the expandable section 24 to rapidly unfurl, moving the microneedle 14 in a substantially perpendicular direction, relative to the longitudinal central axis 12b of the actuator body 12a, to puncture a vascular wall 32a. It may take only between approximately 100 milliseconds and five seconds for the microneedle to move from its furled state to its unfurled state.

The ends of the actuator at the retaining rings or rigid end conditions 22a and 22b remain rigidly fixed to the catheter 20. Thus, they do not deform during actuation. Since the actuator begins as a furled structure, its so-called pregnant shape exists as an unstable buckling mode. This instability, upon actuation, produces a large-scale motion of the microneedle approximately perpendicular to the central axis of the actuator body, causing a rapid puncture of the vascular wall without a large momentum transfer. As a result, a microscale opening is produced with very minimal damage to the surrounding tissue. Also, since the momentum transfer is relatively small, only a negligible bias force is required to hold the catheter and actuator in place during actuation and puncture.

The microneedle, in fact, travels with such force that it can enter sub-epithelial or peri-luminal tissue 32b as well as mucosal, or luminal tissue. Additionally, since the actuator is “parked” or stopped prior to actuation, more precise placement and control over penetration of the lumen wall are obtained.

After actuation of the microneedle and delivery of the drugs to the target region via the microneedle, the activating fluid is exhausted from the open area 26 of the actuator, causing the expandable section 24 to return to its original, furled state. This also causes the microneedle to be withdrawn from the lumen wall. The microneedle, being withdrawn, is once again sheathed by the actuator.

Various microfabricated devices can be integrated into the needle, actuator and catheter for metering flows, capturing samples of biological tissue, and measuring pH. The device 10, for instance, could include electrical sensors for measuring the flow through the microneedle as well as the pH of the pharmaceutical being deployed. The device 10 could also include imaging components, such as an intravascular ultrasonic sensor (IVUS), for locating lumen walls, and fiber optics, as is well known in the art, for viewing the target region. For such complete systems, high integrity electrical, mechanical and fluid connections are provided to transfer power, energy, and pharmaceuticals or biological agents with reliability.

By way of example, the microneedle may have an overall length of between about 200 and 3,000 microns (μm). The interior cross-sectional dimension of the shaft 14b and supply tube 14d may be on the order of 20 to 250 μm, while the tube's and shaft's exterior cross-sectional dimension may be between about 100 and 500 μm. The overall length of the actuator body may be between about 3 and 50 millimeters (mm), while the exterior and interior cross-sectional dimensions of the actuator body can be between about 0.4 and 4 mm, and 0.5 and 5 mm, respectively. The gap or slit through which the central section of the actuator unfurls may have a length of about 4-40 mm, and a cross-sectional dimension of about 100-500 μm. The diameter of the delivery tube for the activating fluid may be about 100 μm. The catheter size may be between 1.5 and 15 French (Fr).

As shown in FIG. 4, the actuator 120 may include a plurality of microneedles 140 and 142 located at different points along a length or longitudinal dimension of the central, expandable section 240. The operating pressure of the activating fluid is selected so that the microneedles move at the same time. Alternatively, the pressure of the activating fluid may be selected so that the microneedle 140 moves before the microneedle 142.

Specifically, the microneedle 140 is located at a portion of the expandable section 240 (lower activation pressure) that, for the same activating fluid pressure, will buckle outwardly before that portion of the expandable section (higher activation pressure) where the microneedle 142 is located. Thus, for example, if the operating pressure of the activating fluid within the open area of the expandable section 240 is two pounds per square inch (psi), the microneedle 140 will move before the microneedle 142. It is only when the operating pressure is increased to four psi, for instance, that the microneedle 142 will move. Thus, this mode of operation provides staged buckling with the microneedle 140 moving at time t1, and pressure p1, and the microneedle 142 moving at time t2 and p2, with t1, and p1, being less than t2 and p2, respectively.

This sort of staged buckling can also be provided with different pneumatic or hydraulic connections at different parts of the central section 240 in which each part includes an individual microneedle.

Also, as shown in FIG. 5, an actuator 220 could be constructed such that its needles 222 and 224A move in different directions. As shown, upon actuation, the needles move at angle of approximately 90° to each other to puncture different parts of a lumen wall. A needle 224B (as shown in phantom) could alternatively be arranged to move at angle of about 180° to the needle 224A.

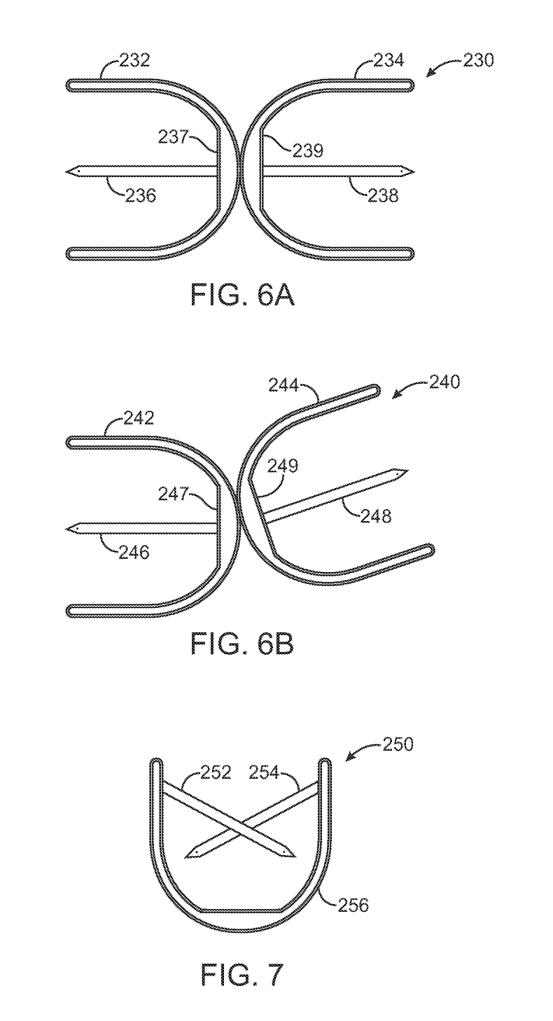

Moreover, as shown in FIG. 6, in another embodiment, an actuator 230 comprises actuator bodies 232 and 234 including needles 236 and 238, respectively, that move approximately horizontally at angle of about 180° to each other. Also, as shown in FIG. 6B, an actuator 240 comprises actuator bodies 242 and 244 including needles 242 and 244, respectively, that are configured to move at some angle relative to each other than 90° or 180°. The central expandable section of the actuator 230 is provided by central expandable sections 237 and 239 of the actuator bodies 232 and 234, respectively. Similarly, the central expandable section of the actuator 240 is provided by central expandable sections 247 and 249 of the actuator bodies 242 and 244, respectively.

Additionally, as shown in FIG. 7, an actuator 250 may be constructed that includes multiple needles 252 and 254 that move in different directions when the actuator is caused to change from the unactuated to the actuated condition. The needles 252 and 254, upon activation, do not move in a substantially perpendicular direction relative to the longitudinal axis of the actuator body 256.

The above catheter designs and variations thereon, are described in published U.S. Patent Application Nos. 2003/005546 and 2003/0055400, the full disclosures of which are incorporated herein by reference. Co-pending application Ser. No. 10/350,314, assigned to the assignee of the present application, describes the ability of substances delivered by direct injection into the adventitial and pericardial tissues of the heart to rapidly and evenly distribute within the heart tissues, even to locations remote from the site of injection. The full disclosure of that co-pending application is also incorporated herein by reference. An alternative needle catheter design suitable for delivering the drug of the present invention will be described below. That particular catheter design is described and claimed in co-pending application Ser. No. 10/393,700 (Attorney Docket No. 021621-001500 U.S.), filed on Mar. 19, 2003, the full disclosure of which is incorporated herein by reference.

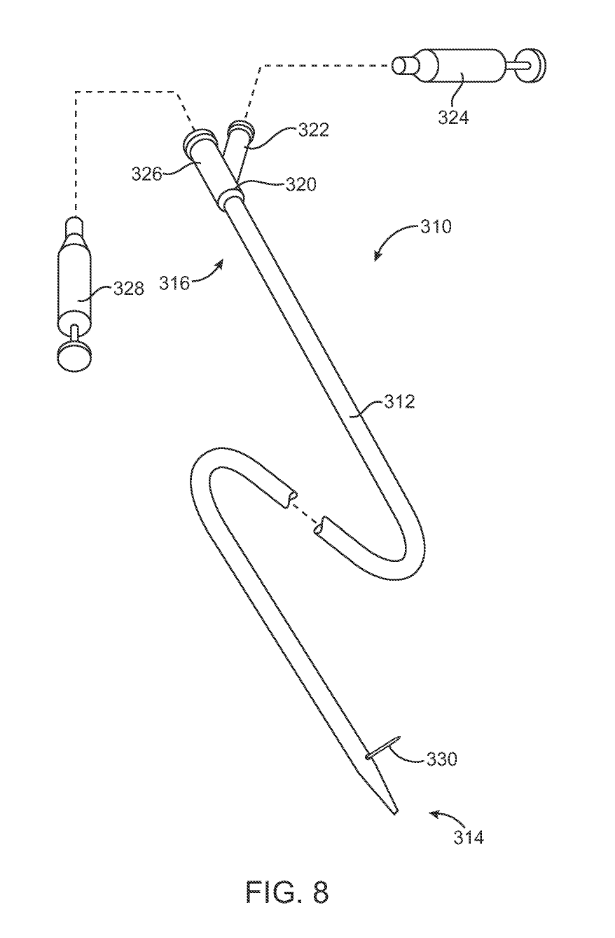

Referring now to FIG. 8, a needle injection catheter 310 constructed in accordance with the principles of the present invention comprises a catheter body 312 having a distal end 314 and a proximal 316. Usually, a guide wire lumen 313 will be provided in a distal nose 352 of the catheter, although over-the-wire and embodiments which do not require guide wire placement will also be within the scope of the present invention. A two-port hub 320 is attached to the proximal end 316 of the catheter body 312 and includes a first port 322 for delivery of a hydraulic fluid, e.g., using a syringe 324, and a second port 326 for delivering the pharmaceutical agent, e.g., using a syringe 328. A reciprocatable, deflectable needle 330 is mounted near the distal end of the catheter body 312 and is shown in its laterally advanced configuration in FIG. 8.

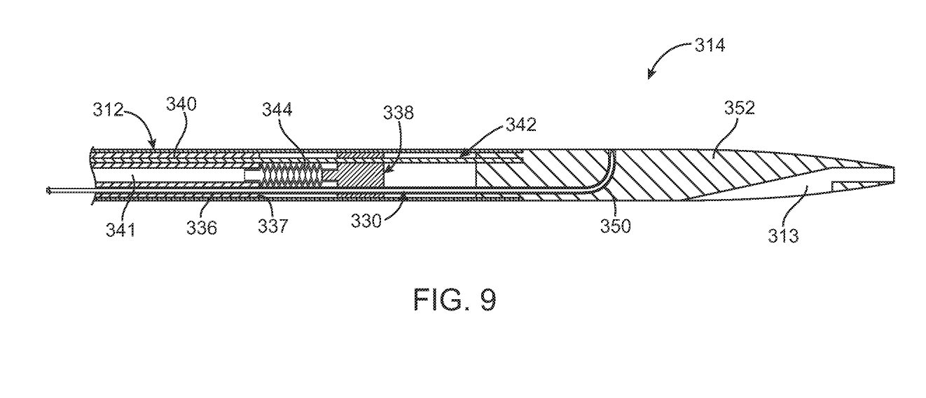

Referring now to FIG. 9, the proximal end 314 of the catheter body 312 has a main lumen 336 which holds the needle 330, a reciprocatable piston 338, and a hydraulic fluid delivery tube 340. The piston 338 is mounted to slide over a rail 342 and is fixedly attached to the needle 330. Thus, by delivering a pressurized hydraulic fluid through a lumen 341 tube 340 into a bellows structure 344, the piston 338 may be advanced axially toward the distal tip in order to cause the needle to pass through a deflection path 350 formed in a catheter nose 352.

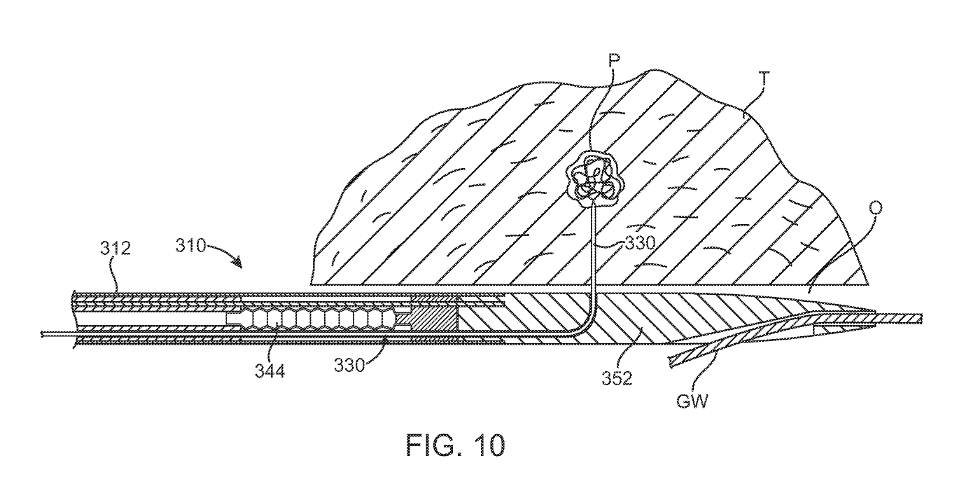

As can be seen in FIG. 10, the catheter 310 may be positioned in a paranasal sinus ostium O, over a guide wire GW in a conventional manner. Distal advancement of the piston 338 causes the needle 330 to advance into sub-epithelial or peri-luminal tissue T adjacent to the catheter when it is present in the sinus. The drug may then be introduced through the port 326 using syringe 328 in order to introduce a plume P of drug in the peri-luminal tissue, as illustrated in FIG. 10.

The needle 330 may extend the entire length of the catheter body 312 or, more usually, will extend only partially in drug delivery lumen 337 in the tube 340. A proximal end of the needle can form a sliding seal with the lumen 337 to permit pressurized delivery of the drug through the needle.

The needle 330 will be composed of an elastic material, typically an elastic or super-elastic metal, typically being nitinol or other super elastic metal. Alternatively, the needle 330 could be formed from a non-elastically deformable or malleable metal which is shaped as it passes through a deflection path. The use of non-elastically deformable metals, however, is less preferred since such metals will generally not retain their straightened configuration after they pass through the deflection path.

The bellows structure 344 may be made by depositing by parylene or another conformal polymer layer onto a mandrel and then dissolving the mandrel from within the polymer shell structure. Alternatively, the bellows 344 could be made from an elastomeric material to form a balloon structure. In a still further alternative, a spring structure can be utilized in, on, or over the bellows in order to drive the bellows to a closed position in the absence of pressurized hydraulic fluid therein.

After the drug is delivered through the needle 330, as shown in FIG. 10, the needle is retracted and the catheter either repositioned for further agent delivery or withdrawn. In some embodiments, the needle will be retracted simply by aspirating the hydraulic fluid from the bellows 344. In other embodiments, needle retraction may be assisted by a return spring, e.g., locked between a distal face of the piston 338 and a proximal wall of the distal tip 352 (not shown) and/or by a pull wire attached to the piston and running through lumen 341.

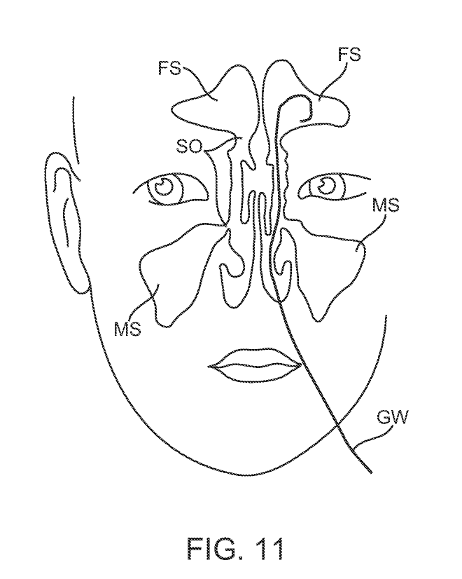

The various methods and devices disclosed herein may be used to deliver one or more substances to various sinus and other cavities in the head and neck, as shown in FIGS. 11-14. Examples of such regions include, but are not limited to paranasal sinuses, Eustachian tubes, middle ear regions, etc. FIG. 11 shows a coronal view of a human head with a guidewire GW placed in a front paranasal sinus FS of a patient, as generally described in U.S. 2006/0106361, the full disclosure of which is incorporated herein by reference. In this example, the guidewire is introduced through a nostril of the patient. The distal end of the guidewire is navigated through the anatomy such that the distal end of the guidewire enters a paranasal sinus. This may be done under fluoroscopic or endoscopic guidance or by other means of guided imaging.

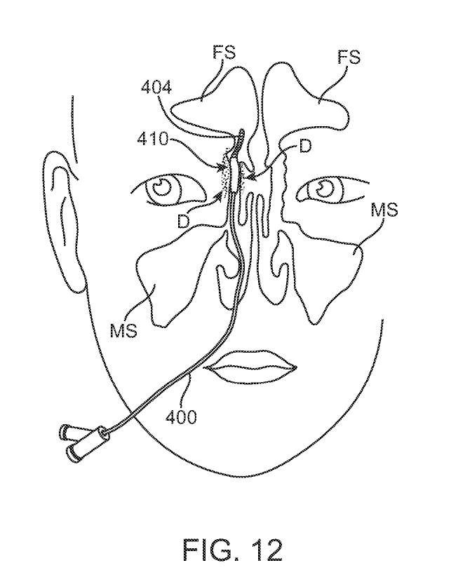

After a guidewire GW is placed as shown in FIG. 11, any of the needle injection/infusion catheters as described in FIGS. 1 through 10 may be introduced over the guidewire. Such a catheter 400 is displayed in FIG. 12.Though a guidewire would be one way to introduce such a catheter, it may not be required, as an endoscopic guide catheter could be placed and the therapeutic catheter strung through the guide catheter, or the therapeutic catheter may have a floppy tip 404 that does not cause any trauma when introduced without a wire or sheath. Furthermore, the needle injection/infusion catheter could then be employed to deliver into the tissue surrounding the paranasal sinuses or other spaces or cavities in the head. Once in place, as in FIG. 12, the needle 410 may be deployed and therapeutic or diagnostic agent D delivered to the sub-epithelial or peri-luminal tissue around the paranasal sinus.

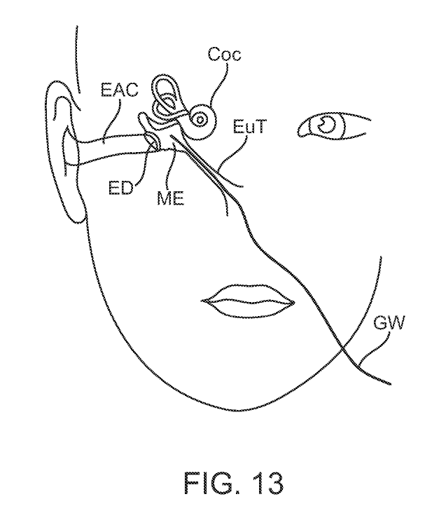

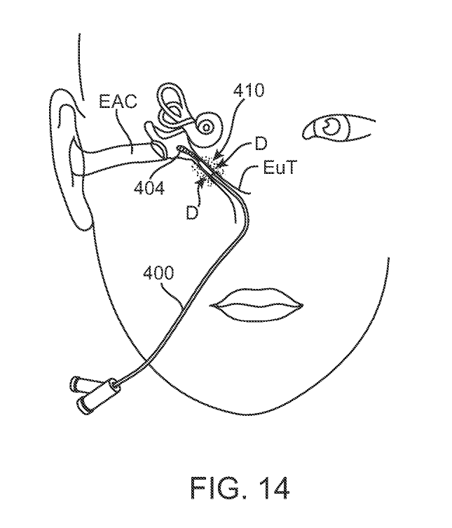

In another embodiment of the present invention, FIG. 13 shows a guidewire GW that may be placed into the Eustachian tube EuT and near the middle ear ME from a nasal approach. In FIG. 13, the external auditory canal EAC may also be a target for intervention, but is not displayed in this rendering. Further, in FIG. 14, the needle injection/infusion catheter 400 utilizing a floppy tip 404 rather than a guidewire or guide catheter may be placed transnasally into the Eustachian tube EuT, at which point the needle 410 may be deployed and therapeutic or diagnostic agent D delivered to the sub-epithelial or peri-luminal tissue.

Another extension of the present application allows for the delivery of drugs through the paranasal sinus lining and into the other recesses of the head, including the brain, ocular cavities, etc. because the paranasal sinus allows direct access to these recesses, providing a needle as described in this application could be used to puncture from the sinuses into these recesses. Applications of stem cells and gene therapy to the base of the brain via a trans-sinus approach is a desirable application of this technology for the treatment of neurodegenerative and other disorders.

All publications, patents, and patent applications cited herein are hereby incorporated by reference in their entirety for all purposes to the same extent as if each individual publication, patent, or patent application were specifically and individually indicated to be so incorporated by reference. While the above is a complete description of the preferred embodiments of the invention, various alternatives, modifications, and equivalents may be used. Therefore, the above description should not be taken as limiting the scope of the invention which is defined by the appended claims.

What is claimed is:

1. A method for treating a body lumen selected from the group consisting of sinus, nasal, pharynx, and Eustachian cavities, said method comprising delivering at least one agent into sub-epithelial or peri-luminal tissue surrounding the body lumen.

2. The method of claim 1, wherein the body lumen comprises a sinus cavity or ostium.

3. The method of claim 2, wherein the sinus cavity comprises a maxillary sinus, a frontal sinus, an ethmoid sinus, or a sphenoidal sinus

4. The method of claim 1, wherein the at least one agent is selected from the group consisting of anti-inflammatory agents, anti-stress agents, antibacterial agents, antifungal agents, antiviral agents, and antiseptics.

5. The method of claim 4, wherein the agent comprises a statin.

6. The method of claim 5, wherein the statin is selected from the group consisting of atorvastin, fluvastatin, lovastatin, mevastatin, pravastatin, rosuvastatin, simvastatin, and any of their derivatives.

7. The method of claim 4, wherein the agent interferes with the action of tumor necrosis factor.

8. The method of claim 7, wherein the agent is selected from the group consisting of etanercept, adalimumab, and infliximab.

9. The method of claim 4, wherein the agent comprises an antibacterial agent.

10. The method of claim 9, wherein the antibacterial agent is selected from the group consisting of aminoglycosides, amphenicols, ansamycins, (3-lactams, lincosamides, macrolides, nitrofurans, quinolones, sulfonamides, sulfones, tetracyclines, and any of their derivatives.

11. The method of claim 10, wherein the antibacterial agent comprises a tetracycline.

12. The method of claim 11, wherein the tetracycline comprises doxycycline.

13. The method of claim 12, wherein doxycycline is administered at a concentration such that local tissue concentrations are obtained which are identical to those achieved with the administration of 20 mg oral equivalent twice a day or less.

14. The method of claim 4, wherein the agent comprises an anti-inflammatory agent.

15. The method of claim 14, wherein the anti-inflammatory agent is a steroid.

16. The method of claim 15, wherein the steroid is selected from the group consisting of triamcinolone, dexamethasone, hydrocortisone, methyl prednisolone, betamethasone.

17. The method of claim 1, wherein the at least one agent is provided in a pharmaceutically acceptable carrier.

18. The method of claim 1, wherein delivering the at least one agent comprises injecting the agent through a mucosa of the body lumen into the sub-epithelial or peri-luminal tissue.

19. A method as in claim 18, wherein the agent is injected to a depth of 0.5 mm to 3 mm beyond a mucosal surface.

20. A method as in claim 1, wherein the agent is delivered to treat a sinus disease.

21. A method as in claim 20, where the sinus disease is rhinitis or sinusitis.

22. A method as in claim 1, wherein the agent is delivered to reduce inflammation.

23. A method as in claim 22, wherein the agent is delivered before, during, or after a sinus procedure that may cause inflammation.

24. A method as in claim 23, wherein the sinus procedure comprises a sinus drainage procedure, a sinus enlargement procedure, a sinus puncture procedure, or an intranasal artostomy.

25. A device for delivering agents across the mucosa of a sinus, sinus ostium, Eustachian tube, or pharynx, said device comprising:
a catheter adapted for insertion into the paranasal sinuses, sinus ostia, Eustachian tube, or pharynx;
a hollow microneedle deployable from the catheter;
wherein the microneedle is adapted to be advanced from the catheter into or through the mucosa and beyond the epithelium for the delivery of therapeutic or diagnostic agents.

26. A method for delivering an agent into the sub-epithelial or peri-luminal tissue surrounding a body lumen selected from the group consisting of a sinus, nasal, pharynx, and Eustacian cavity, said method comprising:
positioning a catheter through a patient's nose or sinusotomy into one of the body lumens;
advancing a needle from the catheter through a mucosal wall into sub-epithelial or peri-luminal tissue surrounding the body lumen; and
delivering the agent into the sub-epithelial or peri-luminal tissue through the needle.

27. A method as in claim 26, wherein the needle is advanced to a depth of 0.5 mm to 3 mm beyond the mucosal surface.
